(19) United States
(12) Patent Application Publication
(10) Pub. No.: US 2026/0150836 A1
(43) Pub. Date: Jun. 4, 2026

(54) DEUTERIUM-LABELLED CANNALACTONE SYNTHESIS AND USE FOR THE DOSAGE OF CANNALACTONE IN ALL TISSUES OR EXUDATES OF LIVING PLANTS OR ORGANISMS

(71) Applicants: CENTRE NATIONAL DE LA RECHERCHE SCIENTIFIQUE (CNRS), PARIS (FR); UNIVERSITE PARIS-SACLAY, GIF-SUR-YVETTE (FR); NANTES UNIVERSITE, NANTES Cedex 1 (FR); HEMP IT ADN, BEAUFORT-EN-ANJOU (FR)

(72) Inventors: François Didier BOYER, Le Chesnay-Rocquencourt (FR); Jean Bernard POUVREAU, Nort sur Erdre (FR); Alexandre MACIUK, Châtenay Malabry (FR); Suzanne DAIGNAN FORNIER DE LACHAUX, Massy (FR); Fabienne MATHIS, Angers (FR)

(21) Appl. No.: 19/256,798

(22) Filed: Jul. 1, 2025

(30) Foreign Application Priority Data
Jul. 1, 2024 (FR) FR2407158

(51) Int. Cl. A01N 43/08; C07B 59/00; C07D 307/58

(52) U.S. Cl. CPC A01N 43/08; C07B 59/002; C07D 307/58; C07B 2200/05

(57) ABSTRACT

The present invention relates to the chemical synthesis of cannalactone, an adaptation of this synthesis leading to deuterium labeling of cannalactone, as well as the use of deuterium-labeled cannalactone to perform the dosage of natural cannalactone of the exudates or tissues of the hemp plant.

### TECHNICAL FIELD OF THE INVENTION

The present invention relates to the chemical synthesis of cannalactone, an adaptation of this synthesis leading to deuterium labeling of cannalactone, as well as the use of deuterium-labelled cannalactone to perform the dosage of cannalactone in all tissues or exudates of living plants or organisms, and in particular in exudates or tissues of the hemp plant by liquid chromatography coupled with mass spectrometry.

### TECHNICAL BACKGROUND

Hemp (Cannabis sativa) is an annual plant originating in Asia and has been used for more than 8,000 years. It is cultivated around the world and is able to cover the four vital needs of humanity: food, housing, clothing, medicine.

Hemp cultivation is widespread because it is a profitable and sustainable production. Indeed, the plant adapts easily to different soils and climates and as such does not require any phytosanitary treatment. The hemp production area in France has increased by a factor of thirty between 1960 and today. This interest in hemp is only increasing, partly with the aim of gradually replacing cotton, which is very water-intensive. France is today the leading hemp producer in Europe with a production of around 20,000 ha.

Known for its resistance to parasites and pests, the hemp plant is all the more attractive. However, a parasitic plant in the Orobanche family, the branched orobanche, Phelipanche ramosa induces large yield losses on hemp crops of up to more than 80%. This parasite also attacks crops such as rapeseed or tobacco, but a specialization of a population of branched hemp orobanche has been brought to light [1]-[3].

After germination, the parasitic plant connects to the root of the host plant and thus recovers nutrients for its own development [4]. This parasitism causes substantial damage to hemp crops.

Strigolactones are small molecules known as being exuded in the soil at picomolar concentrations (10-12 M) and have been identified as stimulants of germination of Phelipanche ramosa or branched orobanche seeds [5], [6]. Strigolactones (SLs) were first identified for their role in parasitic and symbiotic interactions in the rhizosphere and are
the last class of plant hormones to be discovered [7], [8]. They are mostly known for their role in controlling plant architecture, more recently roles for SLs in other aspects of plant development have been brought to light [9].

The Applicant has identified that cannalactone, which is a strigolactone exuded by hemp, was responsible for the initiation of the parasitic life cycle of the branched orobanche [1], [10], [11].

To this end, the Applicant has developed a chemical synthesis allowing access to cannalactone (lowly bioavailable), as well as an adaptation of this synthesis to obtain deuterium-labeled cannalactone and its use to carry out the assay of the natural cannalactone of the exsudates or tissues of the hemp plant.

### SUMMARY OF THE INVENTION

In particular, the present invention relates to deuterium-labeled cannalactone, characterized in that it corresponds to the general Formula 1:

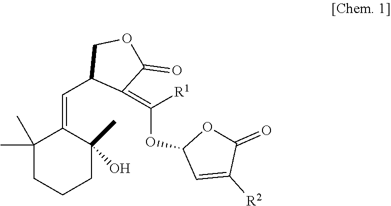

wherein:
R¹ denotes a hydrogen atom H or deuterium atom D,
R² denotes the methyl radical CH₃ or the tri-substituted deuterated methyl radical CD₃,
at least one of R¹ and R² being deuterated.

Advantageously, R¹ may denote a hydrogen atom H and R² may denote the tri-substituted deuterated methyl radical CD₃, so that the deuterium-labeled cannalactone according to the invention will correspond to the following Formula 2:

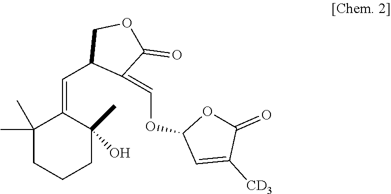

Advantageously, R¹ may denote a deuterium atom D and R² may denote the methyl radical CH₃, so that the deuterium-labeled cannalactone according to the invention will correspond to the following Formula 3:

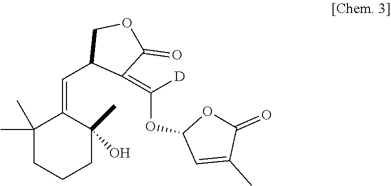

Advantageously, R¹ may denote a deuterium atom D and R² may denote the tri-substituted deuterium methyl radical CD₃, so that the deuterium-labeled cannalactone according to the invention will correspond to the following Formula 4:

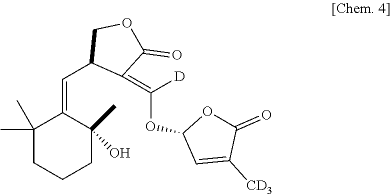

The present invention also relates to a method for synthesizing cannalactone, of the following general Formula 5:

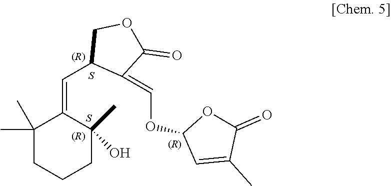

characterized in that it comprises the following steps:
a reaction A) of coupling commercial β-cyclocitral with a bromofuran C4 of the following Formula 6:

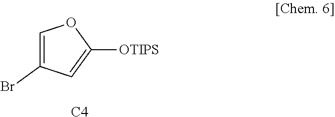

to obtain a B20 alcohol of the following Formula 7: [0027]

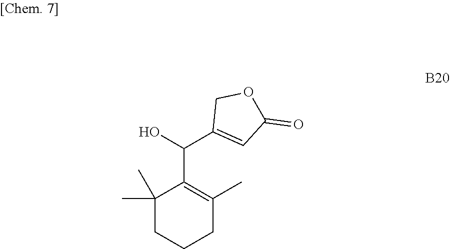 [SMILES: CC1=C(C(O)C2=CC(=O)OC2)C(C)(C)CCC1]

a step B) of reducing the alcohol B20 of Formula 7 in particular by a hydride, to obtain a mixture of allyl alcohol diastereoisomers, followed by a step of separating (in particular by silica chromatography) said diastereoisomers to retain the diastereoisomer (4R*, 6R*)-B21 of the following Formula 8:

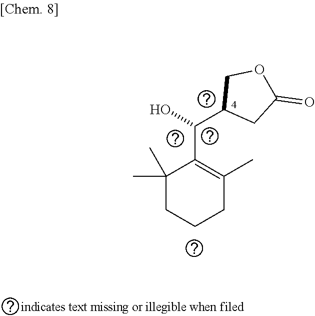

a step C) of selectively epoxidizing the compound (4R*, 6R*)-B21 of Formula 8, in particular directed by the allyl alcohol, to obtain the epoxide (4R*, 6R*)-cis-B22 of the following Formula 9:

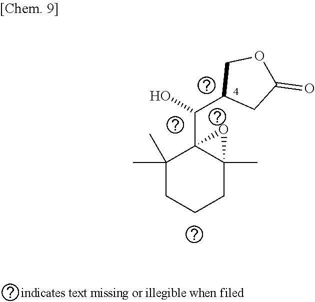

a radical reaction D) for reducing and isomerizing the epoxy (4R*, 6R*)-cis-B22 of Formula 9, to obtain the tertiary alcohol (4R*, 8R*)-B1 of the following Formula 10:

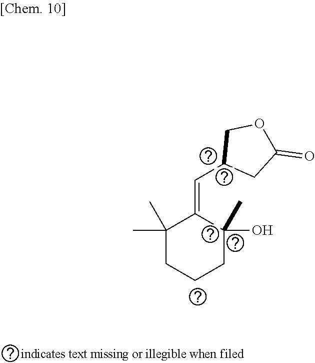

a step E) of protecting the tertiary alcohol (4R*, 8R*-B1 of Formula 10, to obtain the protected alcohol (4R*, 8R*)-B23 of the following Formula 11:

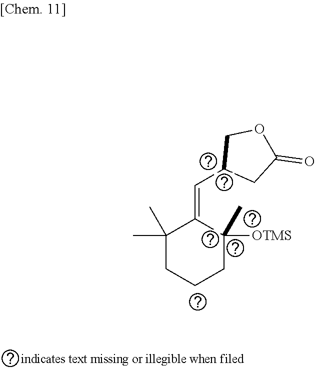

a step F) comprising the formylation F1) of the compound (4R*, 8R*)-B23 of Formula 11 using alkyl formate (in particular ethyl or methyl formate) of the following Formula 12:

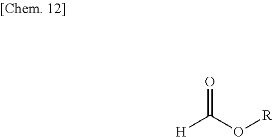

then O-alkylation F2) of the aldehyde thus formed using compound D4 of the following Formula 13:

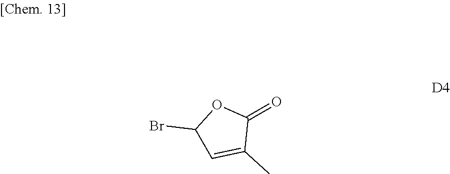

leading to obtaining the diastereoisomer mixture (4R*, 6R*)-B24 of the
following Formula 14:

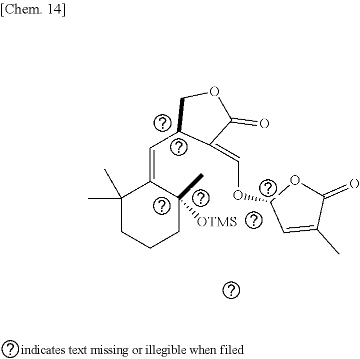

a step G) of deprotecting the tertiary alcohol and separating (in particular by silica chromatography) the diastereoisomers of Formula 14, leading to the isolation of the cannalactone of Formula 5.

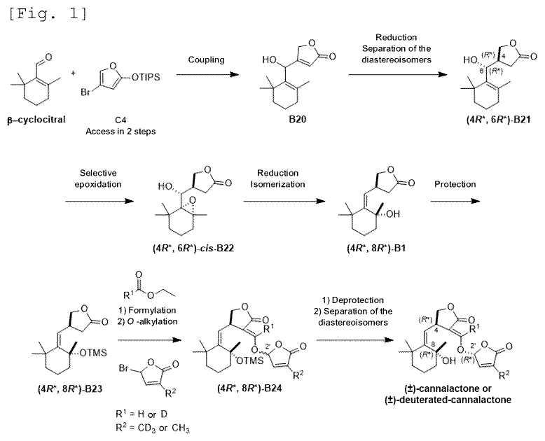

The method for synthesizing cannalactone according to the invention is shown hereinafter in [FIG. 1] which shows the ten-step access route developed by the inventors to chemically synthesize cannalactone.

The present invention also relates to a method for synthesizing a deuterium-labeled cannalactone according to the previously described invention characterized in that it comprises the following steps:
steps A to E of synthesizing the protected compound (4R*, 8R*)-B23 of Formula 11 as described previously for the synthesis of non-deuterated cannalactone (steps A to E are strictly identical);
a step F) comprising formylation F1) of the compound (4R*, 8R*)-B23 of Formula 11 using the alkyl formate (in particular an ethyl or methyl formate) corresponding to Formula 12
or
using deuterated alkyl formate (in particular deuterated ethyl or methyl formate) corresponding to the following Formula 15:

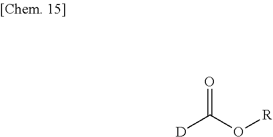

then O-alkylation of the aldehyde thus formed using compound D4 (in particular that described in publication [12]) of the following Formula 13:

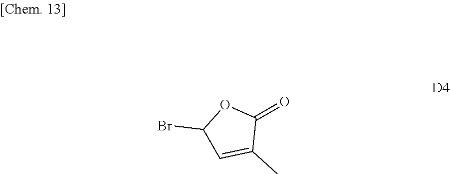

or using the deuterated compound D4 (in particular that described in the publication
[13]) of the following Formula 16:

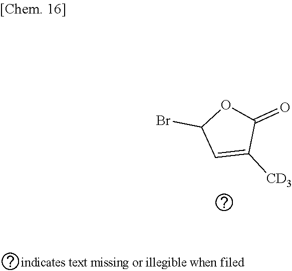 [SMILES: [2H]C([2H])([2H])C1=CC(Br)OC1=O]

at least one of the organic compound functionalized by a formyl group or
compound D4 being deuterated,
to obtain a mixture of deuterated diastereoisomers (4R*, 6R*)-B24 of the following Formula 17:

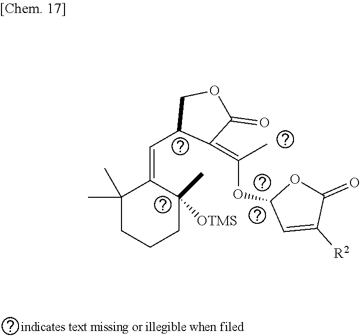

with
R¹ denoting a hydrogen atom H or a deuterium atom D, and
R² denotes a methyl group (CH₃) or a methyl group
where hydrogens have been replaced with deuterium atoms (CD₃),
at least one of R¹ and R² being deuterated;
a step G) of deprotecting the tertiary alcohol and separating the diastereoisomers (in particular by silica chromatography), leading to the isolation of the deuterium-labeled cannalactone of Formula 1.

The method for synthesizing deuterium-labeled cannalactone according to the invention is shown hereinafter also in [FIG. 1]

Advantageously, according to a first advantageous embodiment of the method for synthesizing a deuterium-labeled cannalactone, formylation F1) may be carried out using alkyl formate (in particular ethyl formate or methyl formate) corresponding to Formula 12, and O-alkylation F2) of the aldehyde following formylation F1) may be carried out using compound D4 of Formula 16. In this case, this embodiment will lead to the formation of a deuterium-labeled cannalactone corresponding to Formula 2.

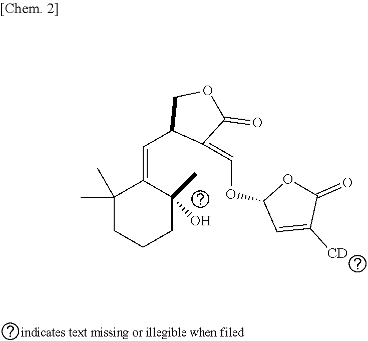

Advantageously, according to a second advantageous embodiment of the method for synthesizing a deuterium-labeled cannalactone, the formylation F1) may be carried out using alkyl formate (in particular ethyl formate or methyl formate) corresponding to Formula 15, and the O-alkylation F2) of the aldehyde after the formylation F1) is carried out using the compound D4 of Formula 13. In this case, this embodiment will lead to the formation of a deuterium-labeled cannalactone corresponding to Formula 3.

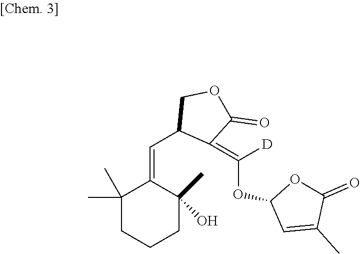

Advantageously, according to a third advantageous embodiment of the method for synthesizing a deuterium-labeled cannalactone, the formylation F1) may be carried out using deuterated alkyl formate (in particular deuterated methyl formate or deuterated ethyl formate) corresponding to Formula 15, and O-alkylation F2) of the aldehyde after formylation F1) is carried out using compound D4 of Formula 16. In this case, this embodiment will lead to the formation of a deuterium-labeled cannalactone corresponding to Formula 4.

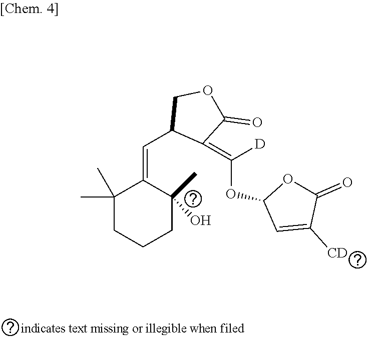

Finally, the present invention further relates to the use of deuterium-labeled cannalactone according to the invention or as obtained according to the method according to the invention for obtaining deuterium-labeled cannalactone, to perform the dosage of cannalactone in any tissue or exudates of living plants or organisms, and in particular in the exudates or tissues of the hemp plant, and in particular to perform the dosage of natural cannalactone of the exudates or tissues of the hemp plant by liquid chromatography coupled with mass spectrometry.

### BRIEF DESCRIPTION OF THE FIGURES

Other characteristics and advantages of the invention may further appear to those skilled in the art upon reading the examples below, given by way of illustration and in a non-limiting manner and shown in the appended [FIG. 1]:

[FIG. 1]-[FIG. 1] shows the diagram of the racemic total synthesis of cannalactone, labeled or not with deuterium.

### EXAMPLES

### Solvents and Reagents

The chemical reagents are commercial products marketed in particular by Sigma Aldrich, Alfa Aesar, Acros Organics and TCI. They were used without additional purification.

The analytical-grade dry solvents are commercial products marketed in particular by Sigma Aldrich and Acros Organics. The tetrahydrofuran (THF) was distilled under argon on sodium in the presence of benzophenone. The deuterated solvents are marketed by Eurisotop.

### Equipment and Methods

The non-aqueous reactions were carried out under an inert atmosphere (argon or nitrogen), using standard techniques for handling compounds sensitive to air and moisture.

All reactions were followed by thin layer chromatography (TLC) on
aluminum plates pre-coated with silica gel (marketed by the company Merck under the trade name 60 F254 with short wavelength UV detection (i.e.) λ=254 nm), and/or by staining with a solution of KMno_{4 [1}% (w/w)] in water or a solution of vanillin [1% (w/w)] in a 1% (v/v) ethanoic solution of phosphoric acid.

Most of the separations were performed under conditions of flash chromatography on silica gel using a filled cartridge (40-63 μm silica gel) at medium pressure (20 psi) with an Armen fraction collector or a pump or a Buchi Pure C-805 Flash.

Some separations were carried out on preparative thin layer chromatography (PTLC) (Merck silica gel 60 F254 on glass).

The ¹H NMR spectra were recorded on Bruker spectrometers at 300, 500
or 700 MHz. The ¹³C NMR spectra were recorded on the same instruments at 75, 125 or 175 MHz. The chemical displacements δ are expressed in parts per million (ppm) with residual solvent signals as internal reference (δ=7.24 for ¹H NMR and 77.23 for ¹³C NMR in CDCl₃). For ¹H NMR, the spectra are described as follows: chemical displacement, integration, multiplicity (s=singlet, d=doublet, t=triplet, q=quadruplet, quint=quintuplet, sext=sixtuplet, dd=doublet of doublet, dt=doublet of triplet, m=multiplet), coupling constant in Herz (J) and attribution. All NMR attributions are based on COSY, HSQC and HMBC experiments. NOESY experiments were recorded to confirm the configurations of the double bonds.

IR spectra were recorded on a PerkinElmer Spectrum 100 FT-IR spectrometer, with absorptions given in centimeters⁻¹ (cm⁻¹).

The low resolution mass spectra were determined by electrospray ionization on a Waters UPLC Acquity system, combined with a photodiode detector (PDA), an evaporative light scattering detector (ELSD), and by a mass spectrometer with a tandem quadrupole detector (TQD). The buffers and aqueous mobile phases for UPLC were prepared using water purified with a Milli-Q system.

The high resolution mass spectra were obtained with the Waters Acquity UPLC device (by direct injection or with a BEH C₁₈ 2.1 Å˜50 mm, 1.7 μm column) combined with a PDA and a Waters LCT Premier XE mass instrument (ESI with a Time of Flight (ToF) analyzer).

Rotational power measurements were recorded on an Anton Paar MCP 300 polarimeter using a 1 dm long cell.

The circular dichroism measurements were recorded on a JASCO J-810 spectropolarimeter at a scan rate of 50 nm/min in acetonitrile from 200 to 400 nm.

The chiral separation of synthetic enantiomers of cannalactone was performed on a THAR supercritical fluid apparatus. This instrument, marketed under the name “Investigator II”, is comprised of a pump module for CO₂ as well as for the co-solvent, a sample changer, a pressure regulator and a collector that can hold up to 6 fractions.

Detection was performed using an iodine band UV spectrometer (marketed under the trade name Waters PDA 2998n from 200 to 800 nm). The instrument parameters have been optimized for the separation of the racemic mixture. The column used is an IC column (cellulose-based immobilized silica protected by tris(3.5-dichlorophenylcarbamate)) from Daicel, size 4.6×250 mm and particle size 5 μm. The furnace temperature was set to 25° C. and the pressure applied to the system was 100 bar. The total flow rate was set to 4 mL/min, with 10% (v/v) methanol. The enantiomers were separated into two bottles using a booster pump at a fixed flow rate of 3 mL/min of methanol. The collection conditions are summarized in Table 1 below:

TABLES 1

| | | Start | Stop | Time | Time |
|---|---|---|---|---|---|
| | Wavelength | threshold | Threshold | window | window |
| | (nm) | (mAU) | (mAU) | Start (min) | Stop (min) |
| Vial 1 | 226 | 20 | 30 | 22.8 | 27.5 |
| Vial 2 | 226 | 20 | 20 | 22.8 | 33 |

### Example 1: Synthesis of Natural Cannalactone in Accordance with the First Method According to the Invention (Access Route Shown in [FIG. 1])

### 4-Bromofuran-2 (5H)-one (C8)

Oxalyl dibromide (2.6 g, 12.00 mmol, 1.2 equiv.) was added to a solution of furan-2.4 (3H, 5H)-dione (1.0 g, 10.00 mmol) in CH₂Cl₂ (22 mL) and DMF (1 mL) at 0° C. The mixture was stirred for 1 h at 0° C. and gradually heated to room temperature for 2 h. The reaction mixture was diluted with water (50 mL) and extracted with EtOAc (3×20 mL). The combined organic phases were washed with water (2×30 mL), a saturated aqueous solution of NaHCO₃ (2×30 mL) and brine (2×30 mL) and dried over Na₂SO₄. The solvents were removed to obtain the crude product C8 (1.61 g, quantitative) in the form of a brown solid. The chemical analyses are in accordance with the literature [14].

### (4-Bromofuran-2-yl)oxytriisopropylsilane (C4)

Et₃N (626.4 mg, 6.20 mmol, 1.4 equiv.) was added to a solution of 4-bromofuran-2 (5H)-one (C8) (720.4 mg, 4.40 mmol) in CH₂Cl₂ (6.2 mL) under argon at 0° C. The mixture was stirred for 1 minute, then triisopropylsilyl trifluoromethanesulfonate (TIPSOTf) (1.42 g, 4.60 mmol, 1.05 equiv.) was added dropwise at 0° C. The mixture thus obtained was stirred for 10 minutes at 0° C., then heated to room temperature and stirred for another 1 h 30. The mixture was diluted with heptane (10 mL), washed with a saturated aqueous solution of NaHCO₃ (2×10 mL), water (2×10 mL) and brine (2×10 mL). The organic phase was dried over Na₂SO₄. The solvents were removed to obtain bromofuran C4 (1.4 g, quantitative) in the form of brown oil. The chemical analyses are in accordance with the literature [14].

### 4-[Hydroxy (8,12,12-trimethylcyclohex-7-en-6-yl)methyl]furan-2 (5H)-one (B20)

A solution of n-BuLi is added dropwise (0.3 mL, 0.30 mmol, 0.98 M, 1.1 equiv.) to a solution of C4 (89.9 mg, 0.28 mmol) in dry THF (1.8 mL) under argon at −78° C. The mixture thus obtained was stirred at −78° C. for 30 minutes. A mixture of β-cyclocitral (51.6 mg, 0.34 mmol, 1.2 equiv.) in dry THF (2 mL) is then added. The reaction mixture was stirred for 2 h at −78° C. and 12 h at room temperature. The mixture was hydrolyzed with a saturated aqueous solution of NH₄Cl (5 mL) and an aqueous solution of HCl (5 mL, 2 M). The organic phase was separated and the aqueous phase was extracted with EtOAc (3×5 mL). The combined organic phases were washed with water (2×5 mL), a saturated aqueous solution of NaHCO₃ (2×5 mL), water (2×5 mL) and brine (2×5 mL), then dried over Na₂SO₄. The solvents were removed and the mixture was purified by silica gel chromatography (heptane/EtOAc, 95:5 to 60:40 for 20 min) to obtain the pure product B20 (24.5 mg, 37%) in the form of brown oil, of Formula 7:

### B20

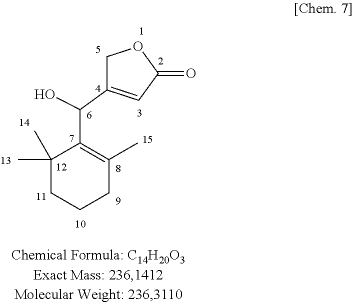 [SMILES: CC1=C(C(O)C2=CC(=O)OC2)C(C)(C)CCC1]

¹H NMR (500 MHZ, CDCl₃) δ 5.91 (1H, d, J=1.5 Hz, H-3), 5.10 (1H, s, H-6), 4.88 (1H, d, J=18.0 Hz, H-5a), 4.71 (1H, d, J=18.0 Hz, H-5b), 1.96 (2H, t, J=6.0 Hz, H-9), 1.61 (3H, s, H-15), 1.59-1.55 (2H, m, H-10), 1.50-1.46 (2H, m, H-11), 1.13 (3H, s, H-13 or H-14), 0.98 (3H, s, H-13 or H-14).

¹³C NMR (75 MHZ, CDCl₃) § 174.1 (C-2), 174.0 (C-4), 138.7 (C-7), 136.6 (C-8), 115.0 (C-3), 71.9 (C-5), 67.8 (C-6), 39.5 (C-11), 35.0 (C-12), 33.7 (C-9), 28.9 (C-13 or C-14), 28.5 (C-13 or C-14), 21.4 (C-15), 19.3 (C-10).

IR (film) ᵥₐₓ 3471, 2932, 1777, 1741, 1637, 1447, 1268, 1111, 1028 cm⁻¹.

HRESIMS m/z 237.1491 [M+H]⁺ (calc, for C₁₄H₂₁O₃, 237.1491).

### 4-[Hydroxy (8,12,12-trimethylcyclohex-7-en-6-yl)methyl]dihydrofuran-2 (3H) one (B21)

To a solution of B20 (696.4 mg, 2.95 mmol) in methanol (45 mL) at 15° C., NiCl₂ (350.9 mg, 1.48 mmol, 0.5 equiv.) is added then sodium borohydride (358.3 mg, 9.47 mmol, 3.2 equiv.) in portions. The mixture was stirred at 15° C. until the TLC analysis showed complete conversion. The reaction mixture was hydrolyzed with an aqueous solution of HCl (50 mL, 2 M). The aqueous phase was extracted with CH₂Cl₂ (3×20 mL). The combined organic phases were dried over Na₂SO₄ and the solvents were removed. The mixture obtained was then purified by silica gel chromatography (CH₂Cl₂/EtOAc, 100:0 to 90:10 for 30 min) to obtain the pure product (4R*, 6R*)-B21 of Formula 8 (327.4 mg, 47%) in the form of yellow oil and (4R*, 6S*)-B21 of Formula 18 (131.9 mg, 19%) in the form of white solid:

### (4R*, 6R*)-B21

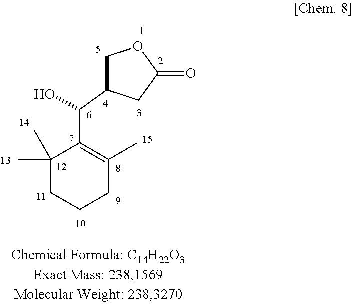

¹H NMR (500 MHZ, CDCl₃) δ 4.50 (1H, dd, J=9.5, 7.0 Hz, H-5a), 4.28 (1H, dd, J=9.5, 7.0 Hz, H-5b), 4.21 (1H, d, J=9.5 Hz, H-6), 3.21 (1H, sext, J=9.5 Hz, H-4), 2.41 (1H, dd, J=17.5, 8.5 Hz, H-3a), 2.18 (1H, dd, J=17.5, 8.5 Hz, H-3b), 1.96 (2H, q, J=5.5 Hz, H-9), 1.81 (3H, s, H-15), 1.59-1.52 (2H, m, H-10), 1.48-1.45 (1H, m, H-11a),

1.40-1.35 (1H, m, H-11b), 1.08 (3H, s, H-13 or H-14), 0.98 (3H, s, H-13 or H-14).

¹³C NMR (125 MHZ, CDCl₃) δ 177.1 (C-2), 138.4 (C-7), 135.1 (C-8), 72.9 (C-5), 72.6 (C-6), 41.3 (C-4), 40.4 (C-11), 35.0 (C-12), 34.6 (C-9), 32.4 (C-3), 29.2 (C-13 or C-14), 29.1 (C-13 or C-14), 21.3 (C-15), 19.4 (C-10).

IR (film) vₘₐₓ 3464, 2928, 1768, 1551, 1365, 1263, 1178, 1048, 1001, 892 cm⁻¹.

HRESIMS m/z 239.1640 [M+H]⁺ (calc, for C₁₄H₂₃O₃, 239.1647).

### (4R*, 6S*)-B21

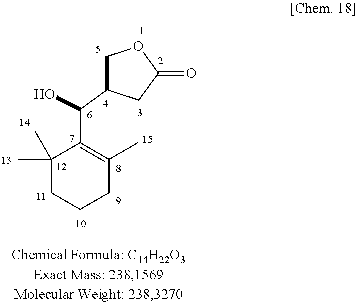

¹H NMR (500 MHZ, CDCl₃) δ 4.17 (1H, dd, J=9.0, 7.0 Hz, H-5a), 4.16 (1H, d, J=9.0 Hz, H-6), 3.94 (1H, dd, J=9.0, 7.0 Hz, H-5b), 3.19 (1H, sext, J=9.0 Hz, H-4), 2.72 (1H, dd, J=17.5, 7.5 Hz, H-3a), 2.57 (1H, dd, J=17.5, 7.5 Hz, H-3b), 1.96 (2H, q, J=5.0 Hz, H-9), 1.80 (3H, s, H-15), 1.59-1.51 (2H, m, H-10), 1.47-1.44 (1H, m, H-11a), 1.40-1.34 (1H, m, H-11b), 1.08 (3H, s, H-13 or H-14), 0.97 (3H, s, H-13 or H-14).

NMR 13C (125 MHZ, CDCl₃) δ 177.5 (C-2), 138.2 (C-7), 135.1 (C-8), 72.3 (C-6), 70.5 (C-5), 41.5 (C-4), 40.4 (C-11), 35.1 (C-12), 34.6 (C-9), 33.8 (C-3), 29.2 (C-13 or C-14), 28.8 (C-13 or C-14), 21.4 (C-15), 19.4 (C-10).

IR (film) vₘₐₓ 3481, 2925, 2870, 1774, 1547, 1465, 1373, 1258, 1176, 1092, 1033, 1011, 890, 795 cm⁻¹.

HRESIMS m/z 239.1638 [M+H]⁺ (calc, for C₁₄H₂₃O₃ 239.1647).

### (4R*)-[(6R*)-hydroxy (8,12,12-trimethyl-7-oxabicyclo[4.1.0]heptan-6yl)methyl]dihydrofuran-2 (3H)-one ((4R*, 6R*)-cis-B22)

A solution of VO(acac) 2 (3.9 mg, 0.015 mmol, 0.04 equiv.) in dry toluene (0.2 mL) was added to a solution of (4R*, 6R*)-B21 (100.9 mg, 0.420 mmol) in dry toluene (5.1 mL). Tert-Butyl hydroperoxide (TBHP) (0.11 mL, 5.5 M, 0.590 mmol, 1.4 equiv.) was then added. The mixture obtained was stirred at room temperature for 1 h. The reaction mixture was hydrolyzed with an aqueous solution of NaOH (5 mL, 5%). The aqueous phase was extracted with heptane and

EtOAc (2:1) (3×10 mL). The combined organic phases were washed with brine (2×10 mL), dried over Na₂SO₄ and the solvents were removed to obtain the pure product (4R*, 6R*)-cis-B22 of Formula 9 (116.4 mg, quantitative) in the form of colorless oil used in the following step without purification:

### (4R*, 6R*)-cis-B22

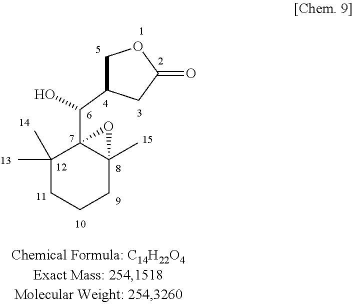

¹H NMR (300 MHZ, CDCl₃) δ 4.40 (1H, dd, J=9.5, 8.0 Hz, H-5a), 4.31 (1H, dd, J=9.5, 8.0 Hz, H-5b), 3.96 (1H, d, J=8.0 Hz, H-6), 2.94 (1H, sext, J=8.0 Hz, H-4), 2.59 (1H, dd, J=17.0, 9.0 Hz, H-3a), 2.48 (1H, dd, J=17.0, 9.0 Hz, H-3b), 1.90-1.80 (1H, m, H-9a), 1.78-1.69 (1H, m, H-9b), 1.39 (3H, s, H-15), 1.36-1.32 (2H, m, H-10), 1.25-1.22 (1H, m, H-11a), 1.06 (3H, s, H-13 or H-14), 1.05-1.03 (1H, m, H-11b), 1.02 (3H,

s, H-13 or H-14).

¹³C NMR (75 MHZ, CDCl₃) δ 176.4 (C-2), 71.1 (C-5), 70.5 (C-6), 70.4 (C-7), 66.3 (C-8), 40.0 (C-4), 37.6 (C-11), 33.9 (C-13), 33.3 (C-3), 31.8 (C-9), 25.6 (C-13 and C-14), 22.2 (C-15), 17.0 (C-10).

IR (film) vₘₐₓ 3464, 2928, 1768, 1551, 1365, 1263, 1178, 1048, 1001, 892 cm⁻¹.

HRESIMS m/z 255.1607 [M+H]⁺ (calc, for C₁₄H₂₃O₄, 255.1596)

### (4R*)-(Z)-[(8R*)-hydroxy-(8,12,12-trimethylcyclohexylidene)methyl]dihydrofuran-2 (3H)-one ((4R*, 8R*)-B1)

A solution of titanocene dichloride (298.7 mg, 1.20 mmol, 5.0 equiv.) and manganese (197.8 mg, 3.60 mmol, 15.0 equiv.) in strictly deoxygenated dry THF (1.2 mL) under argon was stirred for 1 h in a closed flask with a Rodavis stopper. The solution changed from red to green. A solution of (4R*, 6R*)-cis-B22 (61.1 mg, 0.24 mmol) in strictly deoxygenated dry THF (1.2 mL) under argon was added to this mixture. The mixture obtained was stirred for 22 hours at room temperature. The reaction mixture was hydrolyzed with a saturated aqueous solution of NaH₂PO₄ (3 mL). The aqueous phase was extracted with EtOAc (3×5 mL). The organic phase was washed with a saturated aqueous solution of NaHCO₃ (2×5 mL) and brine (2×5 mL), dried with Na₂SO₄ and the solvents were removed. The crude product was purified by silica gel chromatography

### (heptane/EtOAc, 85:15 to 70:30 for 20 min) to obtain a mixture of (4R*, 6R*)-cis-B22, (4R*, 6R*)-B21 and (4R*, 8R*)-B1 (58.2 mg)

The mixture is then diluted in pyridine (0.5 ml) and Ac₂O is added (10 drops). This reaction was stirred for 48 h at room temperature until the TLC analysis indicated complete conversion. The crude product was purified by silica gel chromatography (heptane/EtOAc, 90:10 to 70:30 for 20 min) to obtain the pure product (4R*, 8R*)-B1 of Formula 10 (11.4 mg, 20%) in the form of colorless oil:

### (4R*, 8R*)-B1

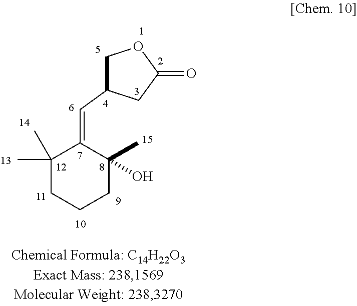

¹H NMR (500 MHZ, CDCl₃) δ 5.25 (1H, d, J=9.5 Hz, H-6), 4.46 (1H, t, J=8.0 Hz, H-5a), 4.30 (1H, sext, J=8.0, H-4), 3.89 (1H, t, J=8.0 Hz, H-5b), 2.61 (1H, dd, J=17.5, 9.0 Hz, H-3a), 2.18 (1H, dd, J=17.5, 9.0 Hz, H-3b), 1.84-1.79 (1H, m, H-9a), 1.59-1.55 (2H, m, H-10), 1.54-1.51 (1H, m, H-9b), 1.42 (3H, s, H-15), 1.39-1.34 (2H, m, H-11), 1.12 (3H, s, H-13 or H-14), 1.05 (3H, s, H-13 or H-14).

¹³C NMR (125 MHZ, CDCl₃) δ 177.7 (C-2), 154.9 (C-7), 124.7 (C-6), 74.5 (C-5), 74.1 (C-8), 44.2 (C-9), 39.7 (C-11), 37.3 (C-12), 36.4 (C-3), 36.0 (C-4), 32.3 (C-13 or C-14), 32.2 (C-13 or C-14), 30.7 (C-15), 19.4 (C-10).

IR (film) vₘₐₓ3490, 2931, 2854, 1775, 1463, 1368, 1256, 1172, 1091, 1007, 872, 788 cm⁻¹.

HRESIMS m/z 239.1639 [M+H]⁺ (calc, for C₁₄H₂₃O₃, 239.1647).

### (4R*)-[(Z)-{(8R*)-(8,12,12-trimethyl-8 [(trimethylsilyl)oxy]cyclohe xylidene)methyl}]dihydrofuran-2 (3H)-one ((4R*, 8R*)-B23)

A mixture of crude alcohol (4R*, 8R*)-B1 (94.3 mg, 0.24 mmol) and trimethylsilylimidazole (TMS-imidazole) (1.06 mL, 7.20 mmol, 30.0 equiv.) was stirred at 50° C. under argon for one night. The mixture was cooled to room temperature, stirred for 1 h and diluted with heptane (5 mL). The organic phase was washed with brine (2×5 mL), dried with Na₂SO₄ and the solvents were removed. The crude product was purified by silica gel chromatography (heptane/EtOAc, 90:10 to 60:40 for 20 min) to obtain the pure product (4R*, 8R*)-B23 of Formula 11 (14.6 mg, 20% in 2 steps) in the form of yellow oil:

### (4R*,8R*)-B23

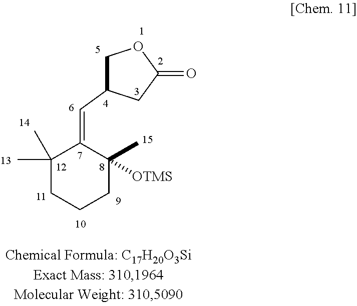

¹H NMR (500 MHZ, CDCl₃) δ 5.18 (1H, d, J=9.5 Hz, H-6), 4.41 (1H, t, J=8.0 Hz, H-5a), 4.31 (1H, sext, J=9.5 Hz, H-4), 3.88 (1H, t, J=8.0 Hz, H-5b), 2.58 (1H, dd, J=17.0, 8.5 Hz, H-3a), 2.15 (1H, dd, J=17.0, 8.5 Hz, H-3b), 1.86-1.81 (1H, m, H-9a), 1.74 (1H, td, J=13.0, 4.5 Hz, H-9b), 1.59-1.53 (2H, m, H-10), 1.42 (3H, s, H-15), 1.40-1.32 (2H, m, H-11), 1.12 (3H, s, H-13 or H-14), 1.04 (3H, s, H-13 or H-14), 0.12 (9H, s, H-TMS).

¹³C NMR (125 MHz, CDCl₃) δ 177.7 (C-2), 155.1 (C-7), 124.1 (C-6), 77.5 (C-8), 74.3 (C-5), 42.6 (C-9), 39.4 (C-11), 37.3 (C-12), 36.3 (C-3), 35.7 (C-4), 32.8 (C-13 or C-14), 32.7 (C-13 or C-14), 32.1 (C-15), 19.3 (C-10), 3.2 (C-TMS).

IR (film) vₘₐₓ 2963, 2928, 1781, 1469, 1366, 1250, 1250, 1162, 1066, 1035, 1012, 838 cm⁻¹. [0138]HRESIMS m/z 311.1952 [M+H]⁺ (calc, for C₁₇H₃₁O₃Si, 311.2042). (4R*, 8R*)-B24

Ethyl formate (32 μL, 0.4 mmol, 10.0 equiv.) and tert-BuOK (32.5 mg, 0.28 mmol, 7.0 equiv.) is added to a solution of (4R*, 8R*)-B23 (11.2 mg, 0.04 mmol) in dry THF (0.4 mL) at −40° C. under argon. The mixture was stirred for 1 h at 0° C. The mixture was diluted in EtOAc (2 mL), washed with water (2×2 mL) and brine (2×2 mL), dried over Na₂SO₄ and concentrated under reduced pressure to obtain the crude enol (7.5 mg, 55%). This compound was used without additional purification in the following step.

Tert-BuOK (3.4 mg, 0.03 mmol, 1.5 equiv.) and a solution of 5bromo-3-methylfuran-2 (5H)-one D4 [12] (5.8 mg, 0.03 mmol, 1.5 equiv.) in dry THF (0.2 mL) were added to a solution of crude enol (7.5 mg, 0.02 mmol) in dry THF (0.2 mL) at −78° C. The reaction medium is heated to room temperature and stirred for one night. The reaction medium was diluted in EtOAc (3 mL), washed with water (2×2 mL) and brine (2×2 mL), then dried with Na₂SO₄ and the solvents were removed. The mixture was purified by PTLC (petroleum ether/EtOAc, 80:20) to obtain the pure product (4R*, 8R*)-B24 of Formula 17 (2.1 mg, 12% in 2 steps) in the form of yellow oil:

### (4R*, 8R*)-B24

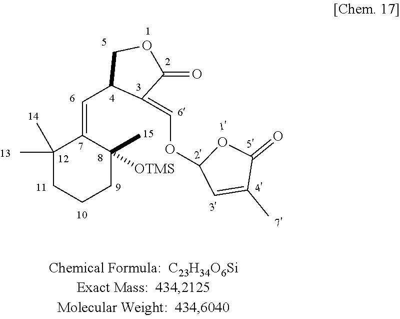

### Isomer 1

¹H NMR (700 MHZ, CDCl₃) δ 7.43 (1H, d, J=3.0 Hz, H-6′), 6.79 (1H, t, J=1.5 Hz, H-3′), 6.06 (1H, s, H-2′), 5.22 (1H, dd, J=13.5, 10.0 Hz, H-6), 4.88-4.84 (1H, m, H-4), 4.47 (1H, dd, J=9.0, 3.0 Hz, H-5a), 3.91 (1H, sext, J=5.5 Hz, H-5b), 1.97 (3H, t, J=1.5 Hz, H-7′), 1.85-1.81 (1H, m, H-11a), 1.73-1.68 (1H, m, H-11b), 1.61-1.54 (2H, m, H-10), 1.46 (3H, s, H-15), 1.39-1.34 (2H, m, H-9), 1.11 (3H, s, H-13 or H-14), 1.08 (3H, s, H-13 or H-14), 0.13 (9H, s, H-TMS).

¹³C NMR (175 MHz, CDCl₃) δ 172.3 (C-2), 170.4 (C-5′), 152.7 (C-7), 150.6 (C-6′), 140.9 (C-3′), 136.0 (C-4′), 124.0 (C-6), 113.3 (C-3), 100.6 (C-2′), 77.7 (C-8), 72.8 (C-5), 43.0 (C-11), 39.7 (C-9), 37.3 (C-12), 37.2 (C-4), 32.8 (C-13 or C-14), 32.7 (C-13 or C-14), 30.9 (C-15), 19.5 (C-10), 10.9 (C-7′), 3.4 (C-TMS).

### Isomer 2

¹H NMR (700 MHZ, CDCl₃) δ 7.45 (1H, d, J=2.5 Hz, H-6′), 6.83 (1H, t, J=1.5 Hz, H-3′), 6.06 (1H, s, H-2′), 5.22 (1H, dd, J=13.5, 10.0 Hz, H-6), 4.88-4.84 (1H, m, H-4), 4.47 (1H, dd, J=9.0, 3.0 Hz, H-5a), 3.91 (1H, sext, J=5.5 Hz, H-5b), 1.95 (3H, t, J=1.5 Hz, H-7′), 1.85-1.81 (1H, m, H-11a), 1.73-1.68 (1H, m, H-11b), 1.61-1.54 (2H, m, H-10), 1.45 (3H, s, H-15), 1.39-1.34 (2H, m, H-9), 1.02 (3H, s, H-13 or H-14), 0.9 (3H, s, H-13 or H-14), 0.12 (9H, s, H-TMS).

¹³C NMR (175 MHZ, CDCl₃) δ 172.3 (C-2), 170.4 (C-5′), 152.7 (C-7), 150.9 (C-6′), 141.0 (C-3′), 135.8 (C-4′), 123.9 (C-6), 113.2 (C-3), 100.7 (C-2′), 77.6 (C-8), 72.6 (C-5), 43.1 (C-11), 39.7 (C-9), 37.3 (C-12), 37.1 (C-4), 32.5 (C-13 or C-14), 32.1 (C-13 or C-14), 30.8 (C-15), 19.5 (C-10), 10.8 (C-7′), 3.4 (C-TMS).

IR (film) vₘₐₓ 2928, 2851, 17887, 1734, 1681, 1463, 1376, 1337, 1250, 1184, 1081, 1031, 1006, 956, 838 cm⁻¹.

HRESIMS m/z 249, 1482 [M+H−H₂ O]⁺ (calc, for C₁₅H₂₁O₃, 249.1491).

### (±)-2′-epi-cannalactone and (±)-cannalactone

A solution of Sc(OTf) 3 (0.3 mg, 7 μmol, 1 mol %) in CH₃CN (0.7 mL) was added to a solution of (4R*, 8R*)-B24 (30.1 mg, 0.070 mmol) in CH₃CN (0.7 mL) and water (5 drops). The resulting mixture was stirred for 1 h 30 at room temperature and hydrolyzed with a phosphate buffer (1.5 mL, pH 7). The organic phase was extracted with CH₂Cl₂ (3×2 mL), and the combined organic phases were washed with brine (2×2 mL), dried over Na₂SO₄, and the solvents were removed to obtain the crude product. The crude product was purified by PTLC (Petroleum ether/EtOAc, 60:40) to obtain the product (±)-2′-epi-cannalactone (8.3 mg, 33% in 3 steps) in the form of yellow oil and (±)-cannalactone of Formula 5 (6.9 mg, 27% in 3 steps) in the form of yellow oil.

### (±)-2′-epi-cannalactone

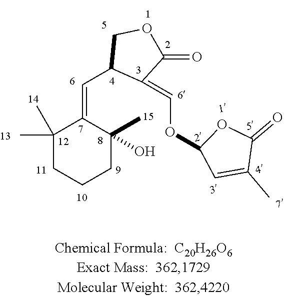

¹H NMR (700 MHZ, CDCl₃) δ 7.43 (1H, d, J=3.0 Hz, H-6′), 6.81 (1H, t, J=1.5 Hz, H-3′), 6.08 (1H, t, J=1.5 Hz, H-2′), 5.32 (1H, d, J=9.5 Hz, H-6), 4.83 (1H, tt, J=9.0, 2.5 Hz, H-4), 4.52 (1H, t, J=9.0 Hz, H-5a), 3.94 (1H, dd, J=9.0, 5.5 Hz, H-5b), 1.98 (3H, t, J=1.5 Hz, H-7′), 1.83-1.79 (1H, m, H-9a), 1.59-1.55 (2H, m, H-10), 1.53-1.48 (1H, m, H-9b), 1.43 (3H, s, H-15), 1.39-1.34 (2H, m, H-11), 1.11 (3H, s, H-13 or H-14), 1.02 (3H, s, H-13 or H-14).

¹³C NMR (175 MHz, CDCl₃) δ 172.3 (C-2), 170.4 (C-5′), 152.5 (C-7), 150.5 (C-6′), 140.9 (C-3′), 136.1 (C-4′), 124.9 (C-6), 113.4 (C-3), 100.5 (C-2′), 73.9 (C-8), 73.2 (C-5), 44.6 (C-9), 40.0 (C-11), 37.3 (C-4), 37.2 (C-12), 32.2 (C-13 or C-14), 31.8 (C-13 or C-14), 29.6 (C-15), 19.6 (C-10), 10.9 (C-7′).

IR (film) vₘₐₓ 3493, 2928, 2848, 1785, 1751, 1684, 1465, 1382, 1347, 1179, 1088, 1031, 954 cm⁻¹. [0156] HRESIMS m/z 363.1813 [M+H]⁺ (calc, for C₂₀H₂₇O₆, 363.1808).

### (±)-cannalactone

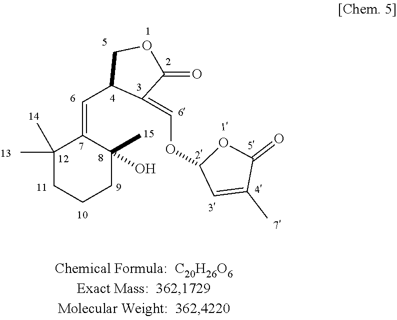

¹H NMR (700 MHZ, CDCl₃) δ 7.47 (1H, d, J=2.5 Hz, H-6′), 6.85 (1H, t, J=1.5 Hz, H-3′), 6.07 (1H, t, J=1.5 Hz, H-2′), 5.31 (1H, d, J=9.5 Hz, H-6), 4.83 (1H, tt, J=9.0, 3.0 Hz, H-4), 4.52 (1H, t, J=9.0 Hz, H-5a), 3.92 (1H, dd, J=9.0, 6.0 Hz, H-5b), 1.95 (3H, t, J=1.0 Hz, H-7′), 1.82-1.78 (1H, m, H-9a), 1.57-1.53 (2H, m, H-10), 1.51-1.47 (1H, m, H-9b), 1.43 (3H, s, H-15), 1.38-1.32 (2H, m, H-11), 1.07 (3H, s, H-13 or H-14), 0.9 (3H, s, H-13 or H-14).

¹³C NMR (175 MHz, CDCl₃) § 172.2 (C-2), 170.4 (C-5′), 152.6 (C-7), 151.1 (C-6′), 141.0 (C-3′), 135.7 (C-4′), 124.5 (C-6), 113.2 (C-3), 100.7 (C-2′), 73.8 (C-8), 72.9 (C-5), 44.6 (C-9), 39.9 (C-11), 37.3 (C-4), 37.1 (C-12), 32.1 (C-13 or C-14), 31.3 (C-13 or C-14), 29.4 (C-15), 19.5 (C-10), 10.7 (C-7′).

IR (film) vₘₐₓ 3479, 2919, 2854, 1784, 1747, 1678, 1466, 1384, 1340, 1182, 1082, 1031, 1007, 951 cm⁻¹.

HRESIMS m/z 363.1794 [M+H]⁺ (calc, for C₂₀H₂₇O₆, 363.1808).
(+)-cannalactone [α]_{D}²⁴+15.00±1.59 (c 0.12, CHCl₃)
(−)-cannalactone [α]_{D}²⁴−11.73±2.53 (c 0.13, CHCl₃)

### Example 2: Synthesis of Deuterium-Labeled Cannalactone in Accordance with the Second Method According to the Invention (Access Route Shown in FIG. 2)

The following products are the same as prepared in Example 1
4-bromofuran-2 (5H)-one (C₈)
(4-bromofuran-2-yl)oxytriisopropylsilane (C₄)
   4-[hydroxy (8,12,12-trimethylcyclohex-7-en-6-yl)methyl]furan-2 (5H)-one (B20)
   4-[hydroxy (8,12,12-trimethylcyclohex-7-en-6-yl)methyl]dihydrofuran-2 (3H)-one (B21)
   (4R*)-[(6R*)-hydroxy (8,12,12-trimethyl-7-oxabicyclo[4.1. 0]heptan-6-yl)methyl]dihydrofuran-2 (3H)-one ((4R*, 6R*)-cis-B22)
   (4R*)-(Z)-[(8R*)-hydroxy-(8,12,12-trimethylcyclohexylidene)methyl]dihydrofuran-2 (3H)-one ((4R*, 8R*)-B1)
   (4R*)-((Z)-[(8R*)-(8,12,12-trimethyl-8 {(trimethylsilyl)oxy}cyclohex ylidene)methyl])dihydrofuran-2 (3H)-one ((4R*, 8R*)-B23).

Ethyl formate (28 μL, 0.35 mmol, 10.0 equiv.) and tert-BuOK (29.8 mg, 0.25 mmol, 7.0 equiv.) were added to a solution of (4R*, 8R*)-B23 of Formula 11 (11.0 mg, 0.035 mmol) in dry THF (0.35 mL) at −40° C. under argon. The mixture was stirred for 1 h at 0° C. The mixture was diluted in EtOAc (2 mL), washed with water (2×2 mL) and brine (2×2 mL), dried with Na₂SO₄ and concentrated under reduced pressure to obtain the crude enol (11.4 mg, 97%).

Tert-BuOK (6.1 mg, 0.05 mmol, 1.5 equiv.) and a solution of deuterated compound D4 [13] (10.2 mg, 0.05 mmol, 1.5 equiv.) in dry THF (0.34 mL) were added to a solution of crude enol (11.4 mg, 0.034 mmol) in dry THE (0.34 mL) at −78° C. The reaction medium is left to warm to room temperature and stirred for one night. The mixture was diluted in EtOAc (3 mL), washed with water (2×2 mL) and brine (2×2 mL), dried with Na₂SO₄, and the solvents were removed to obtain the crude compound (4R*, 8R*)-B24-D3 (21.2 mg) of Formula 17 (alternative wherein R¹ is a hydrogen atom and R² is the CD₃ group):

### (4R*, 8R*)-B24-D3

### [0176] [Chem. 17]

HRESIMS m/z 438, 2393 [M+H]⁺ (calc, for C₂₃H₃₂D₃O₆, 438.2391).

A solution of Sc(OTf) 3 (1.9 mg, 4 μmol, 10 mol %) in CH₃CN (0.34 mL) was added to a solution of (4R*, 8R*)-B24-D3 (14.9 mg, 0.034 mmol) in CH₃CN (0.34 mL) and water (5 drops). The resulting mixture was stirred for 1 h 30 at room temperature and hydrolyzed with a phosphate buffer (1.5 mL, pH 7). The organic phase was extracted with CH₂Cl₂ (3×2 mL), and the combined organic phases were washed with brine (2×2 mL), dried with Na₂SO₄, and the solvents were removed to obtain the crude product. The crude product was purified by silica gel chromatography (petroleum ether/EtOAc, 70:30 to 50:50 for 15 min) to obtain the product (+)-2′-epi-cannalactone-D3 (3.9 mg, 31% in 3 steps) in the form of yellow oil and (+)-cannalactone-D3 of Formula 1 (alternative wherein R¹ is a hydrogen atom and R² is the CD₃ group) (4.1 mg, 32% in 3 steps) in the form of yellow oil.

### Example 3: Biological Results

We performed the dosage of cannalactone in different varieties of hemp using cannalactone according to the invention according to Formulae 2, 3 or 4 according to the protocol described in poi [13]. We were able to quantify it.

### Bibliographic References

1. Hamzaoui, O. et al., Proceedings of the 15th [0180] World Congress on Parasitic Plants; Amsterdam, The Netherlands 32, (2019).
2. Stojanova, B., Delourme, R., Duffé, P., Delavault, P. & Simier, P. Genetic differentiation and host preference reveal non-exclusive host races in the generalist parasitic weed Phelipanche ramosa. Weed Res. 59, 107-118, doi: 10.1111/wre.12353 (2019).
3. Huet, S., Pouvreau, J.-B., Delage, E., Delgrange, S., Marais, C., Bahut, M., Delavault, P., Simier, P. & Poulin, L. Populations of the Parasitic Plant Phelipanche ramosa Influence Their Seed Microbiota. Front. Plant Sci. 11, 1075, doi: 10.3389/fpls.2020.01075 (2020).
4. Delavault, P., Montiel, G., Brun, G., Pouvreau, J. B., Thoiron, S. & Simier, P. Communication Between Host Plants and Parasitic Plants. Adv. Bot. Res. 82, 55-82, doi: 10.1016/bs.abr.2016.10.006 (2017).
5. Cook, C. E.; Whichard, L. P.; Turner, B.; Wall, M. E.; Egley, G. H. Germination of Witchweed (Striga Lutea Lour.): Isolation and Properties of a Potent Stimulant. Science. 154 (3753), 1189-1190. doi: 10.1126/science.154.3753.1189 (1966).
6. Daignan Fornier, S.; Keita, A.; Boyer, F.-D., Chemistry of Strigolactones, Key Players in Plant Communication. ChemBioChem doi: 10.1002/cbic.202400133 (2024).
7. Gomez-Roldan, V., Fermas, S., Brewer, P. B., Puech-Pages, V., Dun, E. A., Pillot, J.-P., Letisse, F., Matusova, R., Danoun, S., Portais, J.-C., Bouwmeester, H., Bécard, G., Beveridge, C. A., Rameau, C. & Rochange, S. F. Strigolactone inhibition of shoot branching. Nature 455, 189-194, doi: 10.1038/nature07271 (2008).
8. Umehara, M., Hanada, A., Yoshida, S., Akiyama, K., Arite, T., TakedaKamiya, N., Magome, H., Kamiya, Y., Shirasu, K., Yoneyama, K., Kyozuka, J. & Yamaguchi, S. Inhibition of shoot branching by new terpenoid plant hormones. Nature 455, 195-200, doi: 10.1038/nature07272 (2008).
9. Lopez-Obando, M., Ligerot, Y., Bonhomme, S., Boyer, F.-D. & Rameau, C. Strigolactone biosynthesis and signaling in plant development. Development 142, 3615-3619, doi: 10.1242/dev.120006 (2015).
10. Daignan Fornier, S. Synthèse totale de la cannalactone, strigolactone non canonique du chanvre et développement d′analogues synthétiques pour leur évaluation biologique. Université Paris-Saclay, (2023).
11. Daignan Fornier, S., de Saint Germain, A., Retailleau, P., Pillot, J.-P., Taulera, Q., Andna, L., Miesch, L., Rochange, S., Pouvreau, J.-B. & Boyer, F.-D. Noncanonical Strigolactone Analogues Highlight Selectivity for Stimulating Germination in Two Phelipanche ramosa Populations. J. Nat. Prod 85, 1976-1992, doi: 10.1021/acs.jnatprod.2c00282 (2022).
12. Macalpine, G. A., Raphael, R. A., Shaw, A., Taylor, A. W. & Wild, H. J. Synthesis of Germination Stimulant (±)-Strigol. J. Chem. Soc., Perkin Trans. 1, 410-416, doi: 10.1039/C39740000834 (1976).
13. Boutet-Mercey, S., Perreau, F., Roux, A., Clave, G., Pillot, J.-P., SchmitzAfonso, I., Touboul, D., Mouille, G., Rameau, C. & Boyer, F.-D.
   Validated
   Method for Strigolactone Quantification by Ultra High-Performance Liquid
   Chromatography—Electrospray Ionisation Tandem Mass Spectrometry Using Novel Deuterium Labelled Standards. Phytochem. Anal. 29, 59-68, doi: 10.1002/pca.2714 (2018).
14. Jas, G. A Simple Resolution of 4-Bromo-2-(Tert-Butyldimethylsiloxy) Furan from Tetrahydro-2,4-Dioxofuran. Synthesis 11, 965-966. doi: 10.1055/s-1991-26618 (1991).

1. A deuterium-labeled cannalactone, wherein the deuterium-labeled cannalactone corresponds to the general Formula (1): 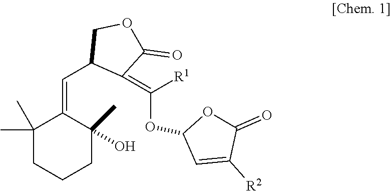
wherein:
R¹ denotes a hydrogen atom H or a deuterium atom D, —R² denotes the methyl radical CH₃ or the trisubstituted deuterated methyl radical CD₃,
at least one of R¹ and R² being deuterated.

2. The deuterium-labeled cannalactone according to claim 1,
wherein: —R¹ denotes H, and
R² denotes CD₃.

3. The deuterium-labeled cannalactone according to claim 1,
wherein: —R¹ denotes D, and
R² denotes CH₃.

4. The deuterium-labeled cannalactone according to claim 1,
wherein: —R¹ denotes D, and
R² denotes CD₃.

5. Method A method of synthesizing cannalactone, of general Formula (5) 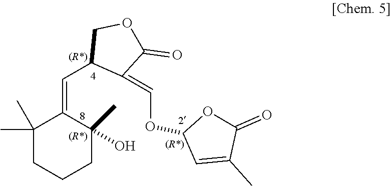
the method comprising:
a reaction A) of coupling commercial β-cyclocitral with unbromofuran C₄ of Formula (6) 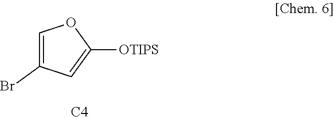
to obtain an alcohol B20 of Formula (7) 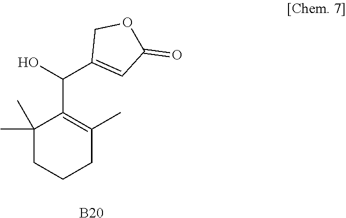
a step B) of reducing the alcohol B20 of Formula (7), to obtain a mixture of diastereoisomers of the allyl alcohol, followed by a step of separating said diastereoisomers to retain the diastereoisomer (4R*, 6R*)-B21 of Formula (8) 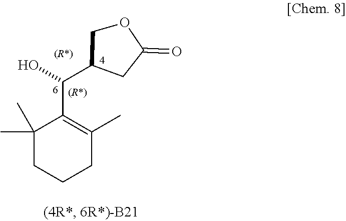
a step C) of selectively epoxidizing the compound (4R*, 6R*)-B21 of Formula (8) to obtain the epoxy 4R*, 6R*)-cis-B22 of Formula (9) 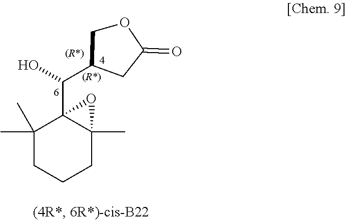
a radical reaction D) of reduction and isomerization of the epoxide (4R*, 6R*)-cis-B22 of Formula (9), to obtain the tertiary alcohol (4R*, 8R*)-B1 of Formula (10) 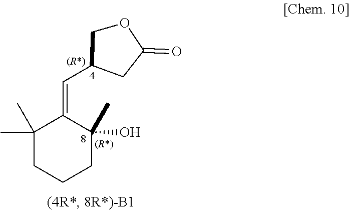
a step E) of protecting the tertiary alcohol (4R*, 8R*)-B1 of Formula (10), to obtain the protected alcohol (4R*, 8R*)-B23 of Formula (11) 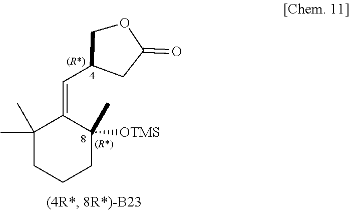
a step F) comprising formylation F1) of the compound (4R*, 8R*)-B23 of Formula (11) using alkyl formate corresponding to Formula (12) 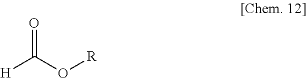
then O-alkylation F2) of the aldehyde thus formed using compound D4 of Formula (13) 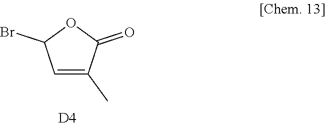
leading to obtaining the diastereoisomer mixture (4R*,6R*) B24 of Formula (14) 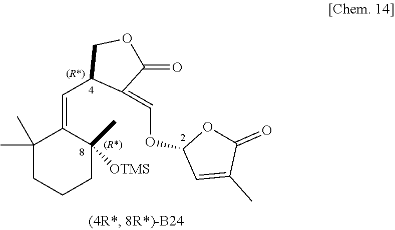
a step G) of deprotecting and separating the diastereoisomers of Formula (14), leading to the isolation of the cannalactone of Formula (5).

6. The method for synthesizing a deuterium-labeled cannalactone as defined according to claim 1, comprising:
synthesizing a protected compound (4R*, 8R*)-B23 of Formula (11) by carrying out
a reaction A) of coupling commercial β-cyclocitral with unbromofuran C4 of Formula (6) 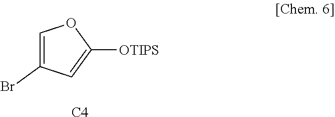
to obtain an alcohol B20 of Formula (7) 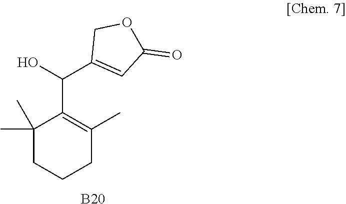
a step B) of reducing the alcohol B20 of Formula (7), to obtain a mixture of diastereoisomers of the allyl alcohol, followed by a step of separating said diastereoisomers to retain the
diastereoisomer (4R*, 6R*)-B21 of Formula (8) 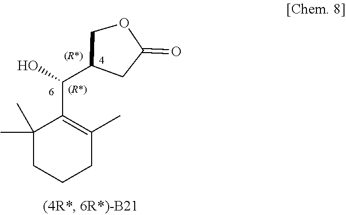
a step C) of selectively epoxidizing the compound (4R*, 6R*)-B21 of Formula (8) to obtain the epoxy 4R*, 6R*)-cis-B22 of Formula
(9) 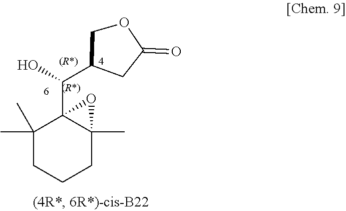
a radical reaction D) of reduction and isomerization of the epoxide (4R*, 6R*)-cis-B22 of Formula (9), to obtain the tertiary alcohol (4R*, 8R*)-B1 of Formula (10) 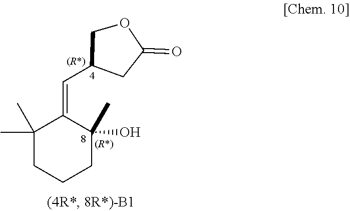
a step E) of protecting the tertiary alcohol (4R*, 8R*)-B1 of Formula (10), to obtain the protected alcohol (4R*, 8R*)-B23 of Formula (11) 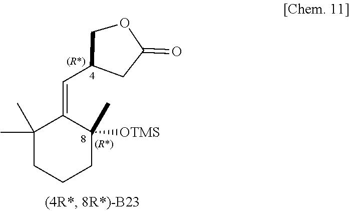
a step F) comprising formylation F1) of the compound (4R*, 8R*)-B23 of Formula (11) using the ethyl formate corresponding to Formula (12)
or
using the deuterated alkyl formate corresponding to Formula (15) 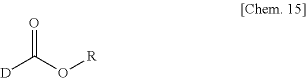
then O-alkylation of the aldehyde thus formed using compound D4 of Formula (13) 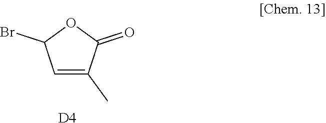
or using the deuterated compound D4 of Formula 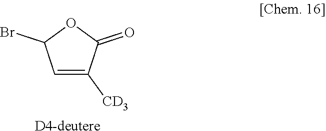 [SMILES: [2H]C([2H])([2H])C1=CC(Br)OC1=O ; "D4-deutere"]
at least one of the organic compound functionalized by a formyl group or the compound D4 being deuterated,
to obtain a mixture of deuterated diastereoisomers (4R*,6R*) B24 of 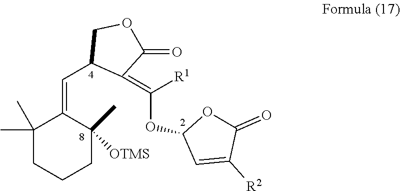
with
R¹ denoting a hydrogen atom H or a deuterium atom D, and
R² denotes a methyl group (CH₃) or a methyl group where the hydrogens have been replaced with deuterium atoms (CD₃);
a step G) of deprotecting and separating the diastereoisomers, leading to the isolation of a deuterium-labeled cannalactone of Formula (1).

7. The method according to claim 6, wherein:
formylation F1) is made of alkyl formate corresponding to Formula (12); and
the O-alkylation F2) of the aldehyde after formylation F1) is carried out using compound D4 of Formula 16.

8. The method according to claim 6, wherein:
formylation F1) is performed using the deuterated alkyl formate corresponding to Formula (15); and
the O-alkylation F2) of the aldehyde after formylation F1) is carried out using compound D4 of Formula (13).

9. The method according to claim 6, wherein:
formylation F1) is performed using the organic compound functionalized by a formyl group corresponding to Formula (15); and
the O-alkylation F2) of the aldehyde after formylation F1) is carried out using compound D4 of Formula (16).

10. A method comprising utilizing the deuterium-labeled cannalactone as defined according to claim 1, to perform the dosage of the cannalactone in any tissue or exudates of living plants or organisms.

11. The method according to claim 10, wherein the method is carried out to perform the dosage of natural cannalactone of the exudates or tissues of the hemp plant by liquid chromatography coupled with mass spectrometry.

12. The method according to claim 10, wherein the tissue or exudates are exudates or tissues of the hemp plant.
